(19) United States
(12) Patent Application Publication
(10) Pub. No.: US 2026/0216524 A1
(43) Pub. Date: Jul. 30, 2026

(54) TERMINATION OF RESUSCITATION ADVIRORY DURING CPR

(71) Applicant: KONINKLIJKE PHILIPS N.V., EINDHOVEN (NL)

(72) Inventors: DAWN BLILIE JORGENSON, Bothell, WA (US); CHENGUANG LIU, Bothell, WA (US); STACY EARL GEHMAN, Bothell, WA (US)

(21) Appl. No.: 19/144,639

(22) PCT Filed: Dec. 21, 2023
(86) PCT No.: PCT/EP2023/087165

§ 371 Date: Jun. 30, 2025

Related U.S. Application Data
(60) Provisional application No. 63/436,526, filed on Dec. 31, 2022.

(51) Int. Cl. A61N 1/39; A61N 1/02; G16H 40/67

(52) U.S. Cl. CPC A61N 1/3925; A61N 1/025; A61N 1/39044; G16H 40/67

(57) ABSTRACT

A defibrillator employing an ECG monitor and a defibrillation controller for supporting a termination of resuscitation (TOR) decision by a responder during a cardiopulmonary resuscitation (CPR) being administered by the responder to a heart of a patient. In operation during the administration of the CPR by the responder to the heart of the patient, the ECG monitor monitors an ECG waveform of the heart of the patient, and the defibrillation controller monitors TOR parameters of the CPR and the ECG waveform, derives a TOR advisory from a monitoring of the TOR parameters, and communicates the TOR advisory to the responder based on at least one of a request by the responder or an occurrence of a TOR event.

### FIELD OF THE INVENTION

The present disclosure generally relates to a cardiac arrest treatment involving cardiopulmonary resuscitation (“CPR”) being administered by a responder to a heart of a patient, and more particularly to supporting a termination of resuscitation (“TOR”) decision during an administration of the CPR by the responder to the heart of the patient.

### BACKGROUND OF THE INVENTION

FIG. 1 illustrates a CPR monitor 30 positioned on the sternum of a patient 10 as a responder 20 applies chest compressions in a conventional manner using two hands with one placed over the other. Instead of placing the hands directly on the patient 10, however, the hands of responder 20 are placed on the CPR monitor 30 and chest compressions are applied to the patient 10 via the CPR monitor 30. Chest compressions are administered by the responder 20 to a heart of patient 10 as prescribed by conventional CPR protocols. As known in the art of the present disclosure, the CPR monitor 30 monitors a quality of the CPR being administered by a responder 20 to a heart of patient 10, such as, for example, whether the CPR is effective or ineffective in terms of a depth and a rate of compression, chest release and recoil, and placement of the responder's hands on the chest of patient 10. A cable 31 is attached to a defibrillator 40 to couple the monitoring of the CPR quality to defibrillator 40 and to issue audible CPR instructions through a loudspeaker of defibrillator 40.

FIG. 1 further illustrates defibrillator 40 attached to patient 10 by electrodes 41a and 41b. Defibrillator 40, as known in the art of the present disclosure, is used to deliver defibrillating shocks to the patient 10 during the CPR as needed. More specifically, defibrillator 40 is operable to deliver a high-voltage impulse to a heart of patient 10 in order to restore normal rhythm and contractile function in patients who are experiencing an arrhythmia (e.g., ventricular fibrillation (VF) or ventricular tachycardia (VT)) that is not accompanied by spontaneous circulation. In operation, defibrillator 20 automatically analyzes an electrocardiogram (ECG) rhythm of the heart of patient 10 to determine if defibrillation is necessary. If so, defibrillator 40 prompts responder 20 to terminate the CPR and to press a shock button to deliver the defibrillation shock to the patient when a shock is advised by defibrillator 40.

The field of resuscitation, as exemplarily shown in FIG. 1, is heavily focused on increasing a quality of care by identifying and providing optimal CPR/shock treatment for a patient experiencing cardiac arrest.

### SUMMARY OF THE INVENTION

The present disclosure is directed to an improvement to existing defibrillators (e.g., Automated External Defibrillators and Advanced Life Support Defibrillators) by providing termination of resuscitation (TOR) advisory in support a TOR decision by a responder during an administration of the cardiopulmonary resuscitation (CPR) by the responder to a heart of a patient.

For purposes of describing and claiming the present disclosure, the term “termination of resuscitation” broadly encompasses criteria for objectively terminating an administration of CPR by a responder to a heart of a patient based on TOR parameters of the CPR and ECG waveform of the patient, and optionally criteria for subjectively terminating the administration of CPR by the responder to the heart of the patient based on the CPR and the ECG waveform of the patient. Examples of objective criteria for TOR during the administration of the CPR include, but are not limited to, (1) an initial or continual unshockable rhythm of the ECG waveform (e.g., asystole), (2) a non-occurrence of a shock delivery in the ECG waveform and (3) a non-occurrence of a return of spontaneous circulation (ROSC) in the ECG waveform.

If the objective criteria is absent, examples of subjective criteria for TOR during the administration of the CPR include, but are not limited to: (1) a duration of the administration of the CPR, (2) a detection or a non-detection of a refractory ventricular fibrillation in the ECG waveform, which can indicate if the patient is likely to have acute coronary occlusions; (3) gasping or non-gasping by the patient, which can be determined from an impedance signal as a sign of a potential effectiveness of the CPR; (4) heart rate at various times during the CPR as an indication of a level of perfusion, particularly an indication of a presence or an absence of a bradycardia rhythm or the potential for some blood flow if the patient was in an organized rhythm for any length of time; and (5) a detection or a non-detection of asystole in the ECG waveform after a shock delivery.

The present disclosure may be embodied as (1) a defibrillator, (2) a defibrillation controller and (3) a defibrillation method.

Various exemplary embodiments of a defibrillator of the present disclosure encompass an ECG monitor and a defibrillation controller for supporting a TOR decision by a responder during an administration of a CPR by the responder to a heart of a patient. The ECG monitor is configured to monitor an ECG waveform of the heart of the patient during the administration of the CPR by the responder to the heart of the patient. The defibrillation controller is configured to, during the administration of the CPR by the responder to the heart of the patient, monitor TOR parameters of the CPR and the ECG waveform, derive a TOR advisory from a monitoring of the TOR parameters, and communicate the TOR advisory to the responder based on a request by the responder or an occurrence of a TOR event.

Various exemplary embodiments of a defibrillation controller of the present disclosure encompass a non-transitory machine-readable storage medium encoded with instructions for execution by one or more processors to support a TOR decision by a responder during an administration of a CPR by the responder to a heart of a patient, the defibrillator. During the administration of the CPR by the responder to the heart of the patient, the non-transitory machine-readable storage medium includes instructions to (1) monitor TOR parameters of the CPR and an ECG waveform of the patient, (2) derive a TOR advisory from a monitoring of the TOR parameters, and (3) communicate the TOR advisory to the responder based on a request by the responder or an occurrence of a TOR event.

Various exemplary embodiments of a defibrillation method in accordance with the present disclosure encompass supporting a TOR decision by a responder during an administration of a CPR by the responder to a heart of a patient. The defibrillation method involves a defibrillator monitoring an ECG waveform of the heart of the patient during the administration of the CPR by the responder to the heart of the patient. The defibrillation method involves the defibrillator, during the administration of the CPR by the responder to the heart of the patient, (1) monitoring TOR parameters of the CPR and the ECG waveform, (2) derive a TOR advisory from a monitoring of the TOR parameters, and (3) communicating the TOR advisory to the responder based on a request by the responder or an occurrence of a TOR event. The foregoing exemplary embodiments and other embodiments of the present disclosure as well as various structures and advantages of the present disclosure will become further apparent to those having ordinary skill in the art from the following detailed description of various embodiments of the present disclosure read in conjunction with the accompanying drawings. The detailed description and drawings are merely illustrative of the present disclosure rather than limiting, the scope of the present disclosure being defined by the appended claims and equivalents thereof.

### BRIEF DESCRIPTION OF THE DRAWINGS

The present disclosure will present in detail the following description of exemplary embodiments with reference to the following figures wherein:

FIG. 1 illustrates a cardiopulmonary resuscitation being administered by a responder to a heart of the patient as known in the art of the present disclosure;

FIG. 2 illustrates an exemplary embodiment of a cardiac arrest treatment system in accordance with the present disclosure;

FIG. 3 illustrates an exemplary embodiment of a defibrillator in accordance with the present disclosure;

FIG. 4 illustrates a flowchart representative of an exemplary embodiment of a defibrillation method in accordance with the present disclosure; and

FIG. 5 illustrates an exemplary embodiment of a defibrillation controller in accordance with the present disclosure.

### DETAILED DESCRIPTION OF THE PREFERRED EMBODIMENTS

The present disclosure is directed to an improvement to existing defibrillators (e.g., Automated External Defibrillators and Advanced Life Support Defibrillators) by providing termination of resuscitation (TOR) advisory in support a TOR decision by a responder during an administration of the cardiopulmonary resuscitation (CPR) by the responder to a heart of a patient.

For purposes of describing and claiming the present disclosure, the term “termination of resuscitation” broadly encompasses criteria for objectively terminating an administration of CPR by a responder to a heart of a patient based on TOR parameters of the CPR and ECG waveform of the patient, and optionally criteria for subjectively terminating the administration of CPR by the responder to the heart of the patient based on the CPR and the ECG waveform of the patient. Examples of objective criteria for TOR during the administration of the CPR include, but are not limited to, (1) an initial or continual unshockable rhythm of the ECG waveform (e.g., asystole), (2) a non-occurrence of a shock delivery in the ECG waveform and (3) a non-occurrence of a return of spontaneous circulation (ROSC) in the ECG waveform.

If the objective criteria is absent, examples of subjective criteria for TOR during the administration of the CPR include, but are not limited to: (1) a duration of the administration of the CPR, (2) a detection or a non-detection of a refractory ventricular fibrillation in the ECG waveform, which can indicate if the patient is likely to have acute coronary occlusions; (3) gasping or non-gasping by the patient, which can be determined from an impedance signal as a sign of a potential effectiveness of the CPR; (4) heart rate at various times during the CPR as an indication of a level of perfusion, particularly an indication of a presence or an absence of a bradycardia rhythm or the potential for some blood flow if the patient was in an organized rhythm for any length of time; and (5) a detection or a non-detection of asystole in the ECG waveform after a shock delivery.

As one having ordinary skill in the art will appreciate in view of the present disclosure, exemplary embodiments of the present invention can be used for providing information to answer a TOR question typically asked of responders, e.g., “was the rhythm shockable at any time?”, and to provide related information in accordance with the present disclosure.

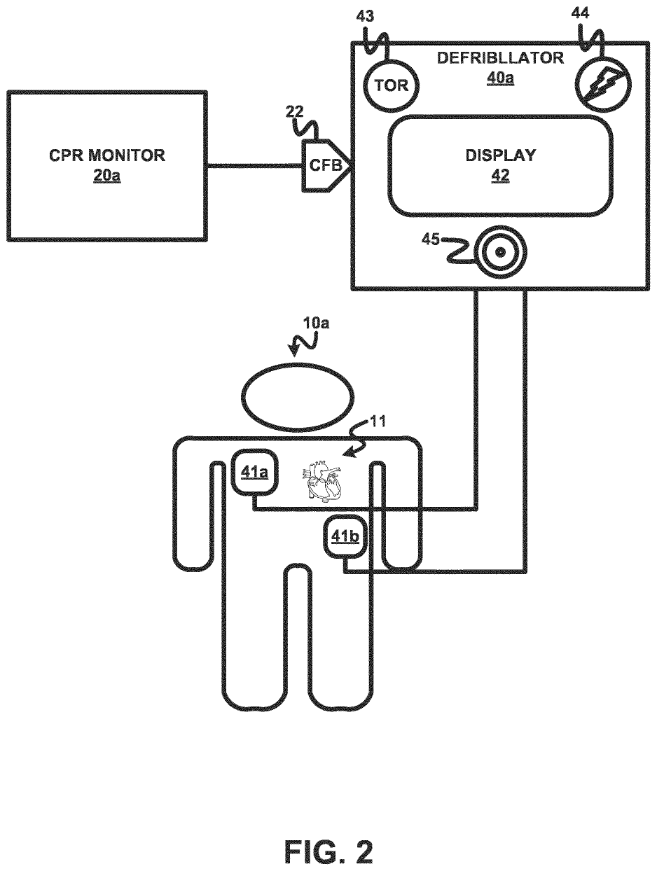

To facilitate an understanding of the present disclosure, the following description of FIG. 2 teaches an exemplary embodiment of a cardiac arrest treatment system in accordance with the present disclosure. From the description of FIG. 2, those having ordinary skill in the art of the present disclosure will appreciate how to apply the present disclosure to make and use additional embodiments of cardiac arrest treatment systems in accordance with the present disclosure.

Referring to FIG. 2, the exemplary cardiac arrest treatment system of the present disclosure employs a CPR monitor 20a and a defibrillator 40a.

In practice, CPR monitor 20a is any device, as known in the art of the present disclosure or hereinafter conceived, for analyzing a quality of CPR being administered by a responder (not shown) to a heart 11 of a patient 10a.

In a first exemplary embodiment, CPR monitor 20a is configured as a CPR coaching device in accordance with U.S. Pat. No. 8,532,765 B2 entitled “CPR Coaching Device with Reduced Sensitivity to Motion” to Ochs et al., the entirety of which is hereby incorporated by reference.

In a second exemplary embodiment, monitor 20a is a CPR mechanical device incorporating the CPR analyzing principles described in Ochs et al.

Still referring to FIG. 2, in practice, defibrillator 40a is any type of defibrillator, as known in the art of the present disclosure or hereinafter conceived, incorporating the inventive principles of the present disclosure for support a TOR decision by a responder during an administration of the cardiopulmonary resuscitation (CPR) by the responder to a heart of a patient.

In operation, defibrillator 40a inputs a CPR feedback 22 from CPR monitor 20a as a basis for monitoring the administration of the CPR by the responder on the heart of the patient.

Further in operation, defibrillator 40a monitors an ECG waveform of heart 11 of patient 10a via electrode 41a and 41b applied to patient 10a as known in the art of the present disclosure, and monitors ECG waveform to detect any shockable rhythm of heart 11 of patient 10a as known in the art of the present disclosure.

If defibrillator 40a detects a shockable rhythm of heart 11 of patient 10a, then defibrillator 40a may communicate a shock indicator to the responder as known in the art of the present disclosure whereby the responder can issue the shock delivery to the heart of the patient. In practice, defibrillator 40a further monitors TOR parameters in the ECG waveform as a basis for ascertaining if the TOR parameters are indicating a TOR objective criteria or a TOR subjective criteria in support of the TOR decision by the responder.

For purposes of describing and claiming the present disclosure, the term “TOR parameters” broadly encompass any parameter, as known in the art of the present disclosure or herein after conceived, that measures whether a cardiac arrest treatment including CPR and shock delivery will likely be successful or unsuccessful.

Examples of TOR parameters include, but are not limited to, rhythm(s) and heartbeats of the ECG waveform, shock indications in the ECG waveform, breathing status of the patient, duration of the CPR.

Still referring to FIG. 2, defibrillator 40a incorporates a use interface including a display 42, a TOR advisory button 43, a shock button 44, and a speaker 45 as means for communicating the TOR advisory to the responder to support a TOR decision by the responder.

For example, if defibrillator 40a determine the TOR parameters are indicating objective TOR criteria, then defibrillator 40a will communicate “a TOR is advisable” to the responder via display 42 and/or speaker 45.

Alternatively by example, if defibrillator 40a determine the TOR parameters are not indicating objective TOR criteria and the responder activates TOR advisory button 43, then defibrillator 40a will communicate subjective TOR criteria indicating a TOR is advisable and subjective TOR criteria indicating a TOR is not advisable to the responder via display 42 and/or speaker 45.

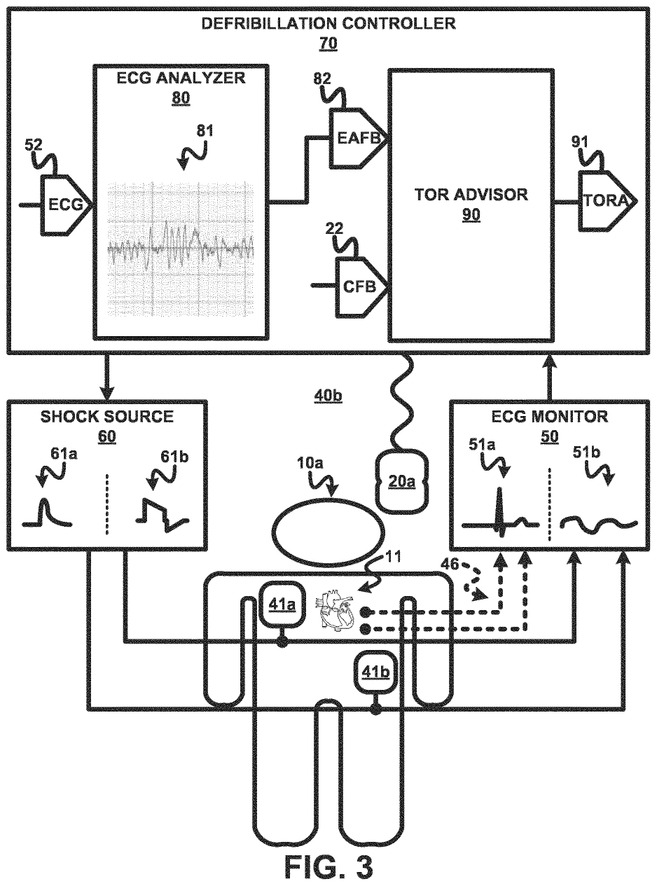

To further facilitate an understanding of the present disclosure, the following description of FIG. 3 teaches an exemplary embodiment of a defibrillator in accordance with the present disclosure. From the description of FIG. 3, those having ordinary skill in the art of the present disclosure will appreciate how to apply the present disclosure to make and use additional embodiments of defibrillator in accordance with the present disclosure.

Referring to FIG. 3, a defibrillator 40b of the present disclosure employs the pair of electrode pads/paddles 41a and 41b, optional ECG leads 46, an ECG monitor 50 (internal or external), a shock source 60, a defibrillation controller 70. Also shown is a CPR coaching device 20a communicatively coupled to defibrillation controller 70.

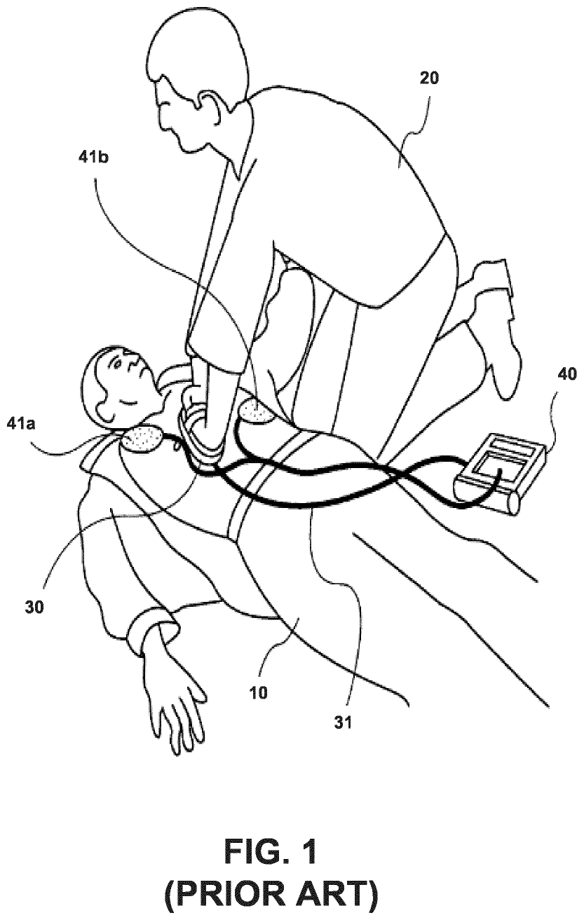

Electrode pads/paddles 41a and 41b are structurally configured as known in the art of the present disclosure to be conductively applied to a patient 10a in an anterior-apex arrangement as shown in FIG. 1 or alternatively in an anterior-posterior arrangement (not shown). Electrode pads/paddles 41a and 41b conduct a defibrillation shock from shock source 60 to heart 11 of patient 10a as controlled by defibrillation controller 70 as known in the art of the present disclosure, and conduct electrical activity of heart 11 of patient 10a to ECG monitor 50 as known in the art of the present disclosure. Alternatively or concurrently, ECG leads 46 as known in the art of the present disclosure may be connected to patient 10a to conduct the electrical activity of heart 11 of patient 10a to ECG monitor 50.

ECG monitor 50 is structurally configured as known in the art to generate an ECG waveform of heart 11 of patient 10a as an indication patient 10a is experiencing an organized heartbeat condition or an unorganized heartbeat condition. An example of ECG waveform indicating an organized heartbeat condition is an ECG waveform 51a as shown in FIG. 3 that is representative of an organized contraction of the ventricles of heart 11 being capable of pumping blood. An example of ECG waveform indicating patient 10a is experiencing an unorganized heartbeat condition is a random ECG waveform 51b as shown in FIG. 3 having zero (0) discernible waves representative of no organized heartbeat activity of heart 11 of patient 10a.

In one exemplary embodiment, ECG monitor 50 employs a digital signal processor (not shown) for streaming ECG waveform data 52 to defibrillation controller 70.

Shock source 60 is structurally configured as known in the art of the present disclosure to store electric energy for delivery of a defibrillation shock via electrode pads/paddles 41a and 41b to heart 11 of patient 10a as controlled by defibrillation controller 70. In practice, the defibrillation shock may have any waveform as known in the art of the present disclosure.

Examples of such waveforms include, but are not limited to, a monophasic sinusoidal waveform (positive sine wave) 61a and a biphasic truncated waveform 61b as shown in FIG. 3.

In one exemplary embodiment, shock source 60 employs a high voltage capacitor bank (not shown) for storing a high voltage via a high voltage charger and a power supply upon a pressing of a charge button. Shock source 60 further employs a switching/isolation circuit (not shown) for selectively applying a specific waveform of an electric energy charge from the high voltage capacitor bank to electrode pads/paddles 41a and 41b as controlled by defibrillation controller 70.

Defibrillation controller 60 incorporates an ECG analyzer 80, as known in the art of the present disclosure and hereinafter conceived, for analyzing and interpreting ECG waveform data 52 from ECG monitor 50.

Defibrillation controller 60 further incorporates a TOR advisor 90 for deriving TOR advisories 91 from ECG feedback data 82 and CPR feedback data 22.

In practice, TOR advisories 91 info the responder whether or not an administration of CPR by the responder should objectively be terminated based on one or more criteria, particularly as set forth by the American Heart Association Examples of standard criteria for objectively terminating the CPR include, (1) an initial or continual unshockable rhythm of the ECG waveform (e.g., asystole), (2) a non-occurrence of a shock delivery in the ECG waveform and (3) a non-occurrence of a return of spontaneous circulation (ROSC) in the ECG waveform.

If the objective criteria is absent, examples of subjective criteria for TOR during the administration of the CPR include, but are not limited to: (1) a duration of the administration of the CPR, (2) a detection or a non-detection of a refractory ventricular fibrillation in the ECG waveform, which can indicate if the patient is likely to have acute coronary occlusions; (3) gasping or non-gasping by the patient, which can be determined from an impedance signal as a sign of a potential effectiveness of the CPR; (4) heart rate at various times during the CPR as an indication of a level of perfusion, particularly an indication of a presence or an absence of a bradycardia rhythm or the potential for some blood flow if the patient was in an organized rhythm for any length of time; and (5) a detection or a non-detection of asystole in the ECG waveform after a shock delivery.

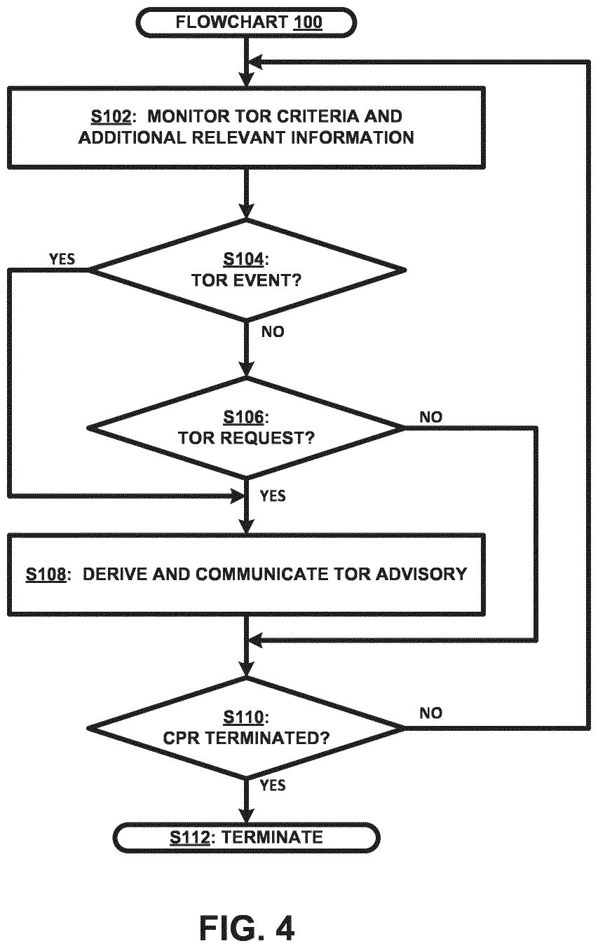

In one embodiment, a flowchart 100 as shown in FIG. 4 is executed by TOR advisor 90 upon a commencement of CPR by the responder.

Referring to FIG. 4, a stage S102 of flowchart 100 encompasses TOR advisor 90 monitoring the TOR parameters of the ECG waveform during the CPR.

Upon an elapse of a specified time, TOR advisor 90 proceeds to a stage S104 of flowchart to ascertain if a TOR event has occurred. For purposes of describing and claiming the present disclosure, the term “TOR event” broadly encompasses a fulfillment of all of the objective TOR criteria for terminating the CPR.

If TOR advisor 90 ascertains that a TOR event has occurred, then TOR advisor proceeds to a stage S110 of flowchart 100 to derive and communicate the TOR advisory of the TOR event to the responder and then proceeds to terminate flowchart 100 if the CPR has been terminated as ascertained by TOR advisor 90 during a stage S110 of flowchart 100.

Otherwise, ff TOR advisor 90 ascertains that a TOR event has not occurred, then TOR advisor proceeds to a stage S116 of flowchart 100 to ascertain if the responder is requesting a TOR advisory.

If TOR advisor 90 ascertains the responder is requesting a TOR advisory, then TOR advisor proceeds to stage S108 of flowchart 100 to derive and communicate the TOR advisory of subjective TPC criteria to the responder and then proceeds to terminate flowchart 100 if the CPR has been terminated as ascertained by TOR advisor 90 during a stage S110 of flowchart 100.

Otherwise, if TOR advisor 90 ascertains the responder is not requesting a TOR advisory, then TOR advisor proceeds to terminate flowchart 100 if the CPR has been terminated as ascertained by TOR advisor 90 during stage S110 of flowchart 100.

In practice, a TOR advisory of a TOR event (i.e., a TOR is advisable) may or may not be followed by the responder. Nonetheless, the present disclosure would have supported this decision by the responder either way during the CPR at a time the ECG waveform is showing continued CPR and shock deliveries will likely not be successful.

Also in practice, a TOR advisory of a subjective TOR criteria (i.e., a TOR may be advisable) facilitates a decision by the responder as to whether or not a TOR is warranted.

Nonetheless, the present disclosure would have supported this decision by the responder during the CPR at a time the ECG waveform is showing continued CPR and shock deliveries may or may not be successful.

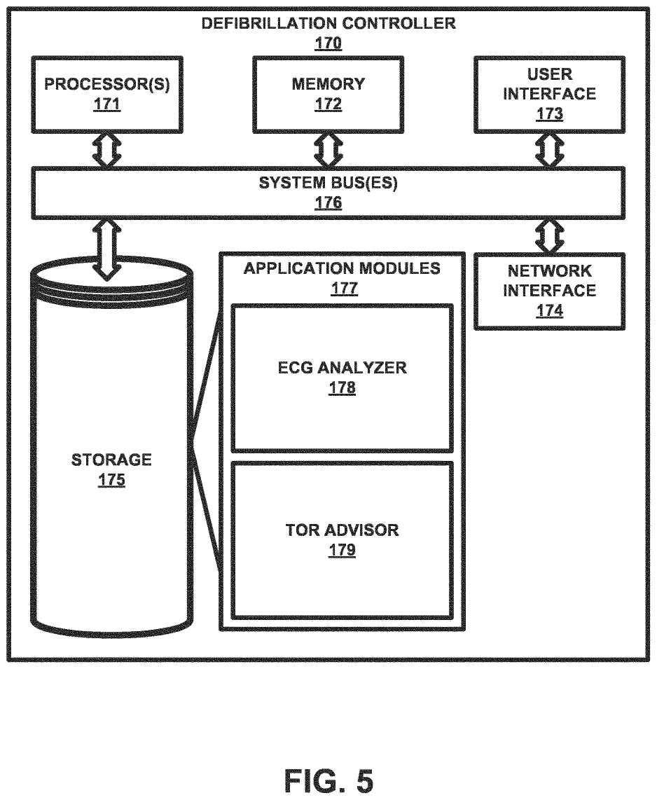

To facilitate a further understanding of the present disclosure, the following description of FIG. 5 teaches an exemplary embodiment of defibrillation controller in accordance with the present disclosure. From the description of FIG. 5, those having ordinary skill in the art of the present disclosure will appreciate how to apply the present disclosure to make and use additional embodiments of a defibrillation controller in accordance with the present disclosure.

Referring to FIG. 5, shown is an exemplary embodiment of defibrillation controller 170 that includes one or more processor(s) 171, memory 172, a user interface 173, a network interface 174, and a storage 175 interconnected via one or more system bus(es) 176.

Each processor 171 can be any hardware device, as known in the art of the present disclosure or hereinafter conceived, capable of executing instructions stored in memory 172 or storage or otherwise processing data. In a non-limiting example, the processor(s) 171 can include a microprocessor, field programmable gate array (FPGA), application-specific integrated circuit (ASIC), or other similar devices.

The memory 172 can include various memories, as known in the art of the present disclosure or hereinafter conceived, including, but not limited to, L1, L2, or L3 cache or system memory. In a non-limiting example, the memory 172 can include static random access memory (SRAM), dynamic RAM (DRAM), flash memory, read only memory (ROM), or other similar memory devices.

The user interface 173 can include one or more devices, as known in the art of the present disclosure or hereinafter conceived, for enabling communication with a user such as an administrator. In a non-limiting example, the user interface can include a command line interface or graphical user interface that can be presented to a remote terminal via the network interface 174.

The network interface 174 can include one or more devices, as known in the art of the present disclosure or hereinafter conceived, for enabling communication other components of a medical device. In a non-limiting example, the network interface 174 can include a network interface card (NIC) configured to communicate according to the Ethernet protocol.

Additionally, the network interface 174 may implement a TCP/IP stack for communication according to the TCP/IP protocols. Various alternative or additional hardware or configurations for the network interface 174 will be apparent.

The storage 175 can include one or more machine-readable storage media, as known in the art of the present disclosure or hereinafter conceived, including, but not limited to, read-only memory (ROM), random-access memory (RAM), magnetic disk storage media, optical storage media, flash-memory devices, or similar storage media. In various non-limiting embodiments, the storage 175 can store instructions for execution by the processor(s) 171 or data upon with the processor(s) 171 may operate. For example, the storage 175 may store a base operating system for controlling various basic operations of the hardware.

The storage 175 can also store an application modules in the form of executable software/firmware for implementing the various functions of the methods of FIGS. 4 and 6 as previously described in the present disclosure.

In one exemplary embodiment as shown, storage 175 stores application modules 177 including an ECG analyzer 178 for monitoring the ECG waveform as known in the art of the present disclosure and TOR advisory 189 for deriving TOR advisories as previously described in the present disclosure, particularly in accordance with flowchart 100 of FIG. 4.

From the description of FIGS. 1-5 herein, those having ordinary skill in the art will appreciate the numerous benefits of the present disclosure including, but not limited to, supporting a termination of resuscitation (TOR) decision by a responder during an administration of the cardiopulmonary resuscitation (CPR) by the responder to a heart of a patient.

The present disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

Further, as one having ordinary skill in the art shall appreciate in view of the teachings provided herein, features, elements, components, etc. disclosed and described in the present disclosure/specification and/or depicted in the appended Figures and/or recited in the Claims can be implemented in various combinations of hardware and software, and provide functions which may be combined in a single element or multiple elements. For example, the functions of the various features, elements, components, etc. shown/illustrated/depicted in the Figures and/or recited in the Claims can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” or “controller” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, combinations thereof, etc.) which is capable of (and/or configurable) to perform and/or control a process.

Moreover, all statements herein reciting principles, aspects, and exemplary embodiments of the present disclosure, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar functionality, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Having described preferred and exemplary embodiments of the present disclosure, which embodiments are intended to be illustrative and not limiting, it is noted that modifications and variations can be made by persons having ordinary skill in the art in view of the teachings provided herein, including the appended Figures and claims. It is therefore to be understood that changes can be made in/to the preferred and exemplary embodiments of the present disclosure which are within the scope of the present disclosure and exemplary embodiments disclosed, described and taught herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A defibrillator for supporting a termination of resuscitation (TOR) decision by a responder during an administration of the cardiopulmonary resuscitation (CPR) by the responder to a heart of a patient, the defibrillator comprising:
an ECG monitor configured to monitor an ECG waveform of the heart of the patient during the administration of the CPR by the responder to the heart of the patient; and
a defibrillation controller, wherein, during the administration of the CPR by the responder to the heart of the patient, the defibrillation controller is configured to:
monitor TOR parameters of the CPR and the ECG waveform;
derive a TOR advisory from a monitoring of the TOR parameters; and
communicate the TOR advisory to the responder based on at least one of a request by the responder or an occurrence of a TOR event.

2. The defibrillator of claim 1, wherein the defibrillation controller is configured to derive the TOR advisory based on the TOR parameters indicating TOR objective criteria of the ECG waveform.

3. The defibrillator of claim 2, wherein the TOR objective criteria include:
an initial or a continual unshockable rhythm of the ECG waveform;
a non-occurrence of a shock delivery in the ECG waveform; and
a non-occurrence of a return of spontaneous circulation in the ECG waveform.

4. The defibrillator of claim 1, wherein the defibrillation controller is configured to derive the TOR advisory based on the TOR parameters indicating TOR subjective criteria of at least one of the ECG waveform and the CPR.

5. The defibrillator of claim 4, wherein the TOR subjective criteria includes at least one of:
a duration of the administration of the CPR;
a detection or a non-detection of a refractory ventricular fibrillation in the ECG waveform;
a gasping or a non-gasping of the patient as indicated by an impedance signal;
a heart rate of the patient in the ECG waveform at various times during the CPR as an indication of a level of perfusion; and
a detection or a non-detection of asystole in the ECG waveform after a shock delivery.

6. A defibrillation controller, comprising:
a non-transitory machine-readable storage medium encoded with instructions for execution by at least one processor to support a termination of resuscitation (TOR) decision by a responder during a cardiopulmonary resuscitation (CPR) being administered by the responder to a heart of a patient,
wherein, during the administration of the CPR by the responder to the heart of the patient, the non-transitory machine-readable storage medium includes the instructions to:
analysis the ECG waveform and derive a TOR advisory from an analysis of the ECG waveform relevant to the TOR decision, and
communicate the TOR advisory to the responder based on at least one of a request by the responder or an occurrence of a TOR event.

7. The defibrillation controller of claim 6, wherein the TOR advisory is based on the TOR parameters indicating TOR objective criteria of the ECG waveform.

8. The defibrillation controller of claim 7, wherein the TOR objective criteria include:
an initial or a continual unshockable rhythm of the ECG waveform;
a non-occurrence of a shock delivery in the ECG waveform; and
a non-occurrence of a return of spontaneous circulation in the ECG waveform.

9. The defibrillation controller of claim 6, wherein the TOR advisory based on the TOR parameters indicating TOR subjective criteria of at least one of the ECG waveform and the CPR.

10. The defibrillation controller of claim 9, wherein the TOR subjective criteria includes at least one of:
a duration of the administration of the CPR;
a detection or a non-detection of a refractory ventricular fibrillation in the ECG waveform;
a gasping or a non-gasping of the patient as indicated by an impedance signal;
a heart rate of the patient in the ECG waveform at various times during the CPR as an indication of a level of perfusion; and
a detection or a non-detection of asystole in the ECG waveform after a shock delivery.

11. A defibrillation method for supporting a termination of resuscitation (TOR) decision by a responder during a cardiopulmonary resuscitation (CPR) being administered by the responder to a heart of a patient, the defibrillator method comprising:
Monitoring, by a defibrillator, an ECG waveform of the heart of the patient during the administration of the CPR by the responder to the heart of the patient; and
during the administration of the CPR by the responder to the heart of the patient:
analyzing, by the defibrillator, the ECG waveform;
deriving, by the defibrillator, a TOR advisory from an analysis of the ECG waveform relevant to the TOR decision, and
communicating, by the defibrillator, the TOR advisory to the responder based on at least one of a request by the responder or an occurrence of a TOR event.

12. The defibrillation method of claim 11, wherein the TOR advisory based on the TOR parameters indicating TOR objective criteria of the ECG waveform.

13. The defibrillation method of claim 12, wherein the TOR objective criteria include:
an initial or a continual unshockable rhythm of the ECG waveform;
a non-occurrence of a shock delivery in the ECG waveform; and
a non-occurrence of a return of spontaneous circulation in the ECG waveform.

14. The defibrillation method of claim 11, wherein the TOR advisory based on the TOR parameters indicating TOR subjective criteria of at least one of the ECG waveform and the CPR.

15. The method of claim 14, wherein the TOR subjective criteria includes at least one of:
a duration of the administration of the CPR;
a detection or a non-detection of a refractory ventricular fibrillation in the ECG waveform;
a gasping or a non-gasping of the patient as indicated by an impedance signal;
a heart rate of the patient in the ECG waveform at various times during the CPR as an indication of a level of perfusion; and
a detection or a non-detection of asystole in the ECG waveform after a shock delivery.
